(19) United States
(12) Patent Application Publication
(10) Pub. No.: US 2022/0298136 A1
(43) Pub. Date: Sep. 22, 2022

(54) PROCESS FOR SYNTHESIS OF PYRAZOLIDINONE COMPOUNDS

(71) Applicant: Gharda Chemicals Limited, Maharashtra (IN)

(72) Inventors: Suchet Saran MATHUR, Maharashtra (IN); Nandkumar Janardan JAIN, Maharashtra (IN); Mahendra Mahipat MORE, Maharashtra (IN)

(21) Appl. No.: 17/636,586

(22) PCT Filed: Aug. 21, 2020
(86) PCT No.: PCT/IB2020/057849

§ 371 Date: Feb. 18, 2022

(30) Foreign Application Priority Data
Aug. 21, 2019 (IN) 201921033691

(51) Int. Cl. C07D 401/04

(52) U.S. Cl. CPC C07D 401/04

(57) ABSTRACT

A process for the synthesis of pyrazolidinone, particularly alkyl 2-(3-chloropyridin-2-yl)-5-oxo-pyrazolidine-3-carboxylate (pyrazolidinone) compound, is provided. The process is simple, economical, and produces alkyl 2-(3-chloropyridin-2-yl)-5-oxo-pyrazolidine-3-carboxylate with a comparatively high yields.

### FIELD

The present disclosure relates to a process for the synthesis of pyrazolidinone compounds.

### BACKGROUND

The background information herein below relates to the present disclosure but is not necessarily prior art.

The pyrazolidinone compounds are used as intermediates for various agrochemicals. Particularly, alkyl 2-(3-chloropyridin-2-yl)-5-oxo-pyrazolidine-3-carboxylate (I) is used as an intermediate for the preparation of agrochemicals such as chlorantraniliprole, cyantraniliprole, etc.

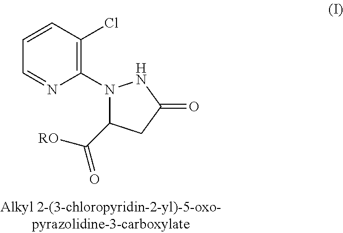

wherein, R is C1-C10 alkyl, benzyl or C3-C5 alkenyl.

Various methods for the preparation of pyrazolidinone compounds are reported in the art.

However, the product obtained by using the conventional methods has a comparatively low yield and low purity.

Therefore, there is felt a need to provide a process for the synthesis of pyrazolidinone that mitigates the aforestated drawbacks.

### OBJECTS

Some of the objects of the present disclosure, which at least one embodiment herein satisfies, are as follows:

It is an object of the present disclosure to ameliorate one or more problems of the prior art or to at least provide a useful alternative.

Another object of the present disclosure is to provide a process for the synthesis of pyrazolidinones.

Yet another object of the present disclosure is to provide a process for the synthesis of pyrazolidinone with a better yield.

Still another object of the present disclosure is to provide a simple and cost effective process for the synthesis of pyrazolidinones.

Other objects and advantages of the present disclosure will be more apparent from the following description, which is not intended to limit the scope of the present disclosure.

### SUMMARY

The present disclosure relates to a process for preparing alkyl 2-(3-chloropyridin-2-yl)-5-oxo-pyrazolidine-3-carboxylate (pyrazolidinone) compounds. The process comprises reacting alkali metal with at least one anhydrous aliphatic alcohol having a carbon atom C1-C5, by optionally heating at a temperature in the range of 50° C. to 100° C. to obtain an alkali metal alkoxide. Hydrazine compound is reacted with the alkali metal alkoxide in the presence of organo silver complex at a temperature in the range of 20° C. to 50° C. to obtain a reaction mixture. A dialkyl ester of carboxylic acid is reacted with the reaction mixture at a temperature in the range of 20° C. to 50° C. to obtain a reaction mass comprising alkyl 2-(3-chloropyridin-2-yl)-5-oxo-pyrazolidine-3-carboxylate (pyrazolidinone) compound. The alkyl 2-(3-chloropyridin-2-yl)-5-oxo-pyrazolidine-3-carboxylate (pyrazolidinone) compound is separated from the reaction mass.

### DETAILED DESCRIPTION

Embodiments, of the present disclosure, will now be described herein. Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

The pyrazolidinone compounds are used as intermediates for various agrochemicals. Particularly, alkyl 2-(3-chloropyridin-2-yl)-5-oxo-pyrazolidine-3-carboxylate (I) is used as an intermediate for the preparation of agrochemicals such as chlorantraniliprole, cyantraniliprole, etc.

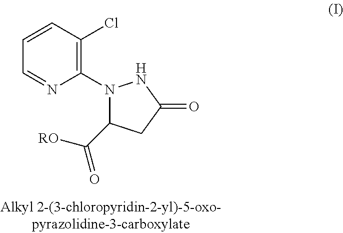

wherein, R is C1-C10 alkyl, benzyl or C3-C5 alkenyl

The process of the present disclosure provides a simple, environment friendly and economical process that results in improved yields and higher purity of the final product.

In an aspect of the present disclosure, there is provided a process for the synthesis of pyrazolidinone compounds. Particularly, the present disclosure provides a process for the preparation of alkyl 2-(3-chloropyridin-2-yl)-5-oxo-pyrazolidine-3-carboxylate compounds.

The process is described in detail.

In a first step, alkali metal is reacted with at least one anhydrous aliphatic alcohol having a carbon atom C1-C5 by optionally heating at a temperature in the range of 50° C. to 100° C. to obtain an alkali metal alkoxide.

In accordance with one embodiment, at least one anhydrous aliphatic alcohol having carbon atom C1-C5 is selected from the group consisting of methanol, ethanol, n-propanol, isopropanol, n-butanol, and iso-butanol.

In an embodiment, when the anhydrous aliphatic alcohol is isopropanol, n-butanol, a mixture of alkali metal and the alcohol is heated to reflux at a temperature in the range of 50° C. to 100° C. In another embodiment when the anhydrous aliphatic alcohol is methanol or ethanol, heating of a mixture of alkali metal and the alcohol is not required.

The so obtained alkali metal alkoxide is cooled to a temperature in the range of 20° C. to 50° C., prior to addition of hydrazine compound.

In an embodiment, the alkali metal is selected from the group consisting of lithium, sodium, potassium, and caesium. In an exemplary embodiment, the alkali metal is sodium.

In a second step, a hydrazine compound is reacted with the alkali metal alkoxide in the presence of organo silver complex at a temperature in the range of 20° C. to 50° C. to obtain a reaction mixture. The reaction is carried out under continuous stirring.

In an exemplary embodiment, the hydrazine compound is 3-chloro-2-hydrazinopyridine.

In an embodiment, the organo silver complex is used as a catalyst. The organo silver complex is a coordination compound of silver salt and at least one ligand. The silver salt is selected from the group consisting of Silver (I) halides, Silver (I) nitrate, and Silver acetate. In an embodiment, the silver halide is selected from the group consisting of Silver iodide, Silver chloride, Silver bromide and Silver fluoride.

In an embodiment, the ligand is at least one selected from the group consisting of trimethylphosphine, triethylphosphine, tris(tert-butyl)phosphine, tricyclopentylphosphine, tricyclohexylphosphine (PCy3), tri(methylcyclohexyl)phosphine, methyl(tetramethylene)phosphine, tert-butyl(pentamethylene)phosphine, triphenylphosphine (PPh3), tri(methylphenyl)phosphine, 1,2-diphenylphosphinecyclohexane, 1,2-diphenylphosphinecyclopentane, 2,2′-(diphenylphosphine)-biphenyl, 1,2-bis(diphenylphosphine)ethane, 1,3-bis(diphenylphosphine)propane, 1,4-bis(diphenylphosphine)butane, 3,4-bis(diphenylphosphine)pyrrolidine, 2,2′-(diphenylphosphine)-bisnaphthyl (Binap), 1,1′-bis(diphenylphosphine)fenocene, 1,1′-bis(di-tert-butylphosphine)ferrocene and diphenyl ether bisdiphenylphosphine.

In an embodiment, the organo silver complex is at least one selected from the group consisting of Tris(triphenylphosphine)Silver(I) chloride, Tris(triphenylphosphine)Silver(I) bromide, Tris(triphenylphosphine)Silver(I) Iodide, Tris(triphenylphosphine)Silver(I) nitrate, Bis(triphenylphosphine)Silver(I) chloride, Bis(triphenylphosphine)Silver(I) bromide, Bis(triphenylphosphine)Silver(I) Iodide, Bis(triphenylphosphine)Silver(I) nitrate Monotriphenyl phosphine monoiodosilver(I), Monotriphenyl phosphine monobromosilver(I), Monotriphenyl phosphine monochloro silver(I) and Monotriphenyl phosphine mononitro silver(I). In an exemplary embodiment, the organosilver complex is Tris(triphenylphosphine)Silver(I) chloride, Tris(triphenylphosphine)Silver(I) bromide, Tris(triphenylphosphine)Silver(I) Iodide, Tris(triphenylphosphine)Silver(I) nitrate.

In a third step, dialkyl ester of carboxylic acid is reacted with the reaction mixture by slowly adding the dialkyl ester of carboxylic acid to the reaction mixture at a temperature in the range of 20° C. to 50° C. to obtain a reaction mass comprising alkyl 2-(3-chloropyridin-2-yl)-5-oxo-pyrazolidine-3-carboxylate (pyrazolidinone) compound. The reaction is carried out under continuous stirring.

In an embodiment, dialkyl ester of carboxylic acid is selected from the group consisting of fumarate ester, maleate ester and a mixture thereof. In an exemplary embodiment, the dialkyl ester of carboxylic acid is e selected from the group consisting of diethyl maleate, diisopropyl fumarate, diisopropyl maleate, and di-n-butyl maleate.

In an embodiment, dialkyl ester of carboxylic acid is slowly added to the reaction mixture over a time period in the range of 30 minutes to 150 minutes.

In an embodiment, the reaction mass so obtained is equilibrated at 20° C. to 50° C. and monitored by HPLC. The reaction is terminated and worked up when optimum formation of pyrazolidinone compound is observed.

Finally, the so obtained alkyl 2-(3-chloropyridin-2-yl)-5-oxo-pyrazolidine-3-carboxylate compound is separated from the reaction mass to obtain alkyl 2-(3-chloropyridin-2-yl)-5-oxo-pyrazolidine-3-carboxylate (pyrazolidinone) compound having a purity greater than 96% and a yield greater than 80%.

Therefore, the process of the present disclosure provides a higher yield of the product with greater purity while also being cost efficient and economical.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results can be extrapolated to industrial scale.

### Experimental Details

### EXAMPLE 1

750 ml of Isopropanol (1.5 litres/mole) was charged into the reactor followed by the addition of sodium pieces (13.8 gms; 1.2 gm atom/mole) and heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of Ag(PPh₃)₃1 catalyst (0.103 gm; 0.0002 mole/mole) under stirring at 25° C. to obtain a reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 84% and purity was 97%.

### EXAMPLE 2

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of Ag(PPh₃)₃Br catalyst (0.0974 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 83% and purity was 96.8%.

### EXAMPLE 3

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain sodium slurry containing isopropoxide. 3-chloro-2-hydrazinopyridine (71.75 gm; 0.50 mole) was added to the slurry containing sodium isopropoxide, followed by the addition of Ag(PPh₃)₃C1 catalyst (0.093 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 80% and purity was 96.3%.

### EXAMPLE 4

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by addition of Ag(PPh₃)₃NO₃ catalyst (0.096 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 80% and purity was 96.4%.

### EXAMPLE 5

190 ml of n-butanol (0.76 litres/mole) was charged into the reactor followed by the addition of sodium pieces (6.9 gms; 1.20 gm atom/mole) under stirring and then heated to 80° C. for complete dissolution to obtain solution containing sodium n-butoxide. The solution was cooled to 30° C. to obtain slurry containing sodium n-butoxide. 3-chloro-2-hydrazinopyridine (36 gm; 0.25 mole) was added to the slurry containing sodium n-butoxide, followed by addition of Ag(PPh₃)₂1 catalyst (0.0765 gm; 0.0003 mole/mole) under stirring at 30° C. to obtain reaction mixture. Di-n-butyl maleate (68.4 gms; 1.20 mole/mole) was added slowly to the reaction mixture over a period of 1 hour keeping reaction temperature between 28-32° C. to obtain a reaction mass. The reaction mass was equilibrated at 28-32° C. and monitored by HPLC. The reaction was terminated and worked up when optimum formation of n-butyl 2-(3-chloropyridin-2-yl)-5-oxo-pyrazolidine-3-carboxylate was observed. The yield of n-butyl 2-(3-chloropyridin-2-yl)-5-oxo-pyrazolidine-3-carboxylate was 80% and purity was 97%.

### EXAMPLE 6

375 ml of absolute Ethanol (0.750 litres/mole) was charged into the reactor followed by the addition of sodium pieces (12.65 gms; 1.1 gm atom/mole) under stirring till dissolution to obtain a solution containing sodium ethoxide. 3-chloro-2-hydrazinopyridine (71.75 gm; 0.50 mole) was added to the solution containing sodium ethoxide, followed by the addition of Ag(PPh₃)₃1 catalyst (0.1021 gm; 0.0002 mole/mole) under stirring at 30° C. to obtain reaction mixture. Di ethyl maleate (103.2 gms; 1.20 mole/mole) was added slowly to the reaction mixture over a period of 2 hours keeping reaction temperature between 30-32° C. to obtain a reaction mass. The reaction mass was equilibrated at 30-32° C. and monitored by HPLC. The reaction was terminated and worked up when optimum formation of Ethyl-2-(3-chloropyridin-2-yl)-5-oxo-pyrazolidine-3-carboxylate was observed. The yield of Ethyl-2-(3-chloropyridin-2-yl)-5-oxo-pyrazolidine-3-carboxylate was 81% and purity was 96.1%.

### EXAMPLE 7

750 ml of Isopropanol (1.5 litres/mole) was charged into the reactor followed by the addition of sodium pieces (12.65 gms; 1.1 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by addition of Ag(PPh₃)₃1 catalyst (0.103 gm; 0.0002 mole/mole) under stirring at 25° C. to obtain reaction mixture. Di-isopropyl fumarate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 83% and purity was 97.4%.

### COMPARATIVE EXAMPLES:

### EXAMPLE 8

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by addition of AgI catalyst (0.0235 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 51% and purity was 95%.

### EXAMPLE 9

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of AgBr catalyst (0.0188 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 45% and purity was 95%.

### EXAMPLE 10

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of AgCl catalyst (0.0143 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 45% and purity was 95%.

### EXAMPLE 11

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of AgNO₃ catalyst (0.017 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate is 40% and purity was 93%.

### EXAMPLE 12

750 ml of Isopropanol (1.5 litres/mole) was charged into the reactor followed by the addition of sodium pieces (13.8 gms; 1.2 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of Ag(COOCH₃) catalyst (0.0167 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 35% and purity was 92%.

### EXAMPLE 13

190 ml of n-butanol (0.76 litres/mole) was charged into the reactor followed by the addition of sodium pieces (6.9 gms; 1.20 gm atom/mole) under stirring and then heated to 80° C. for complete dissolution to obtain solution containing sodium n-butoxide. The solution was cooled to 30° C. to obtain slurry containing sodium n-butoxide. 3-chloro-2-hydrazinopyridine (36 gm; 0.25 mole) was added to the slurry containing sodium n-butoxide, followed by addition of AgI catalyst (0.0176 gm; 0.0003 mole/mole) under stirring at 30° C. to obtain reaction mixture. Di-n-butyl maleate (68.4 gms; 1.20 mole/mole) was added slowly to the reaction mixture over a period of 1 hour keeping reaction temperature between 28-32° C. to obtain a reaction mass. The reaction mass was equilibrated at 28-32° C. and monitored by

HPLC. The reaction was terminated and worked up when optimum formation of n-butyl 2-(3-chloropyridin-2-yl)-5-oxo-pyrazolidine-3-carboxylate was observed. The yield of n-butyl 2-(3-chloropyridin-2-yl)-5-oxo-pyrazolidine-3-carboxylate was 50% and purity was 95.7%.

### EXAMPLE 14

375 ml of absolute Ethanol (0.750 litres/mole) was charged into the reactor followed by the addition of sodium pieces (12.65 gms; 1.1 gm atom/mole) under stirring till dissolution to obtain solution containing sodium ethoxide. 3-chloro-2-hydrazinopyridine (71.75 gm; 0.50 mole) was added to the solution containing sodium ethoxide, followed by the addition of AgI catalyst (0.0235 gm; 0.0002 mole/mole) under stirring at 30° C. to obtain reaction mixture. Di ethyl maleate (103.2 gms; 1.20 mole/mole) was added slowly to the reaction mixture over a period of 2 hours keeping reaction temperature between 30-32° C. to obtain a reaction mass. The reaction mass was equilibrated at 30-32° C. and monitored by HPLC. The reaction was terminated and worked up when optimum formation of Ethyl- 2-(3-chloropyridin-2-yl)-5-oxo-pyrazolidine-3-carboxylate was observed. The yield of Ethyl- 2-(3-chloropyridin-2-yl)-5-oxo-pyrazolidine-3-carboxylate was 46% and purity was 96%.

### EXAMPLE 15

750 ml of Isopropanol (1.5 litres/mole) was charged into the reactor followed by the addition of sodium pieces (12.65 gms; 1.1 gm atom/mole) and then heated to a reflux temperature (80° C. to 85° C.) under stirring till dissolution to obtain a solution containing sodium isopropoxide. The solution was cooled to 25° C. to obtain slurry containing sodium isopropoxide. 3-chloro-2-hydrazinopyridine (71.75 gm; 0.50 mole) was added to the slurry containing sodium isopropoxide, followed by the addition of AgI catalyst (0.0235 gm; 0.0002 mole/mole) under stirring at 25° C. to obtain reaction mixture. Di-isopropyl maleate (125 gms; 1.25 mole/mole) was added slowly to the reaction mixture over a period of 2 hours keeping reaction temperature between 25-30° C. to obtain a reaction mass. The reaction mass was equilibrated at 25-30° C. and monitored by HPLC. The reaction was terminated and worked up when optimum formation of Isopropyl 2-(3-chloropyridin-2-yl)-5-oxo-pyrazolidine-3-carboxylate was observed. The yield of Isopropyl 2-(3-chloropyridin-2-yl)-5-oxo-pyrazolidine-3-carboxylate was 51% and purity was 95%.

The results of Examples 1-15 are summarized in table 1 below:

TABLE-1
SUMMARY OF EXPERIMENTAL RESULTS

| | | | |
|---|---|---|---|
| Example No. | Catalyst | Yield (%) | Purity (%) |

Examples in accordance with the present disclosure

| | | | |
|---|---|---|---|
| 1 | Ag(PPh3)3I | 84 | 97 |
| 2 | Ag(PPh3)3Br | 83 | 96.8 |
| 3 | Ag(PPh3)3Cl | 80 | 96.3 |
| 4 | Ag(PPh3)3NO3 | 80 | 96.4 |
| 5 | Ag(PPh3)2I | 80 | 97 |
| 6 | Ag(PPh3)3I | 81 | 96.1 |
| 7 | Ag(PPh3)3I | 83 | 97.4 |

Comparative Examples

| | | | |
|---|---|---|---|
| 8 | AgI | 51 | 95 |
| 9 | AgBr | 45 | 95 |
| 10 | AgCl | 45 | 95 |
| 11 | AgNO3 | 40 | 93 |
| 12 | Ag(COOCH3) | 35 | 92 |
| 13 | AgI | 50 | 95.7 |
| 14 | AgI | 46 | 96 |
| 15 | AgI | 51 | 95 |

It is evident from Table-1 that organo silver complexes (with ligands) exhibited higher yield and higher purity of pyrazolidinone compounds when compared to the silver salts.

### Technical Advancement

The present disclosure described herein above has several technical advantages including, but not limited to, the realization of a process for synthesis of pyrazolidinone compounds which is simple, economical and results in higher yield and higher purity of the product.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values given for various physical parameters, dimensions, and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A process for preparing alkyl 2-(3-chloropyridin-2-yl)-5-oxo-pyrazolidine-3-carboxylate (pyrazolidinone) compound represented by a formula (I): 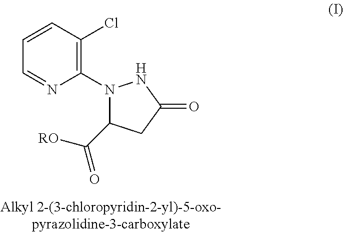
wherein, R represents C1to C10 alkyl, benzyl or C3 to C5 alkenyl;
said process comprising the following steps:
a) reacting alkali metal with at least one anhydrous aliphatic alcohol having a carbon atom C1-C5 by optionally heating at a temperature in the range of 50° C. to 100 ° C. to obtain an alkali metal alkoxide;
b) reacting a hydrazine compound with said alkali metal alkoxide in the presence of organo silver complex at a temperature in the range of 20° C. to 50° C. to obtain a reaction mixture;
c) reacting dialkyl ester of carboxylic acid with said reaction mixture by slowly adding said dialkyl ester of carboxylic acid to said reaction mixture at a temperature in the range of 20 to 50° C. to obtain a reaction mass comprising alkyl 2-(3-chloropyridin-2-yl)-5-oxo-pyrazolidine-3-carboxylate (pyrazolidinone) compound; and
d) separating alkyl 2-(3-chloropyridin-2-yl)-5-oxo-pyrazolidine-3-carboxylate compound from said reaction mass to obtain alkyl 2-(3-chloropyridin-2-yl)-5-oxo-pyrazolidine-3-carboxylate (pyrazolidinone) compound.

2. The process as claimed in claim 1, wherein said anhydrous aliphatic alcohol is at least one selected from the group consisting of methanol, ethanol, n-propanol, isopropanol, n-butanol, and iso-butanol.

3. The process as claimed in claim 1, wherein said alkali metal is selected from the group consisting of lithium, sodium, potassium, and caesium.

4. The process as claimed in claim 1, wherein said alkali metal is sodium.

5. The process as claimed in claim 1, wherein said hydrazine compound is 3-chloro-2-hydrazinopyridine.

6. The process as claimed in claim 1, wherein said alkali metal alkoxide is cooled to a temperature in the range of 20° C. to 50° C. prior to addition of hydrazine in step b).

7. The process as claimed in claim 1, wherein said organo silver complex is a coordination compound of silver salt and at least one ligand.

8. The process as claimed in claim 7, wherein said silver salt is selected from the group consisting of Silver (I) halides, Silver (I) nitrate, and Silver acetate.

9. The process as claimed in claim 7, wherein said ligand is selected from the group consisting of trimethylphosphine, triethylphosphine, tris(tert-butyl)phosphine, tricyclopentylphosphine, tricyclohexylphosphine (PCy3), tri(methylcyclohexyl)phosphine, methyl(tetramethylene)phosphine, tert-butyl(pentamethylene)phosphine, triphenylphosphine (PPh3), tri(methylphenyl)phosphine, 1,2-diphenylphosphinecyclohexane, 1,2-diphenylphosphinecyclopentane, 2,2′-(diphenylphosphine)-biphenyl, 1,2-bis(diphenylphosphine)ethane, 1,3-bis(diphenylphosphine)propane, 1,4-bis(diphenylphosphine)butane, 3,4-bis(diphenylphosphine)pyrrolidine, 2,2′-(diphenylphosphine)-bisnaphthyl (Binap), 1,1′-bis(diphenylphosphine)ferrocene, 1,1′-bis(di-tert-butylphosphine)ferrocene and diphenyl ether bisdiphenylphosphine.

10. The process as claimed in claim 1, wherein said organo silver complex is selected from Tris(triphenylphosphine)Silver(I) chloride, Tris(triphenylphosphine)Silver(I) bromide, Tris(triphenylphosphine)Silver(I) Iodide, Tris(triphenylphosphine)Silver(I) nitrate Bis(triphenylphosphine)Silver(I) chloride, Bis(triphenylphosphine)Silver(I) bromide, Bis(triphenylphosphine)Silver(I) Iodide, Bis(triphenylphosphine)Silver(I) nitrate Monotriphenyl phosphine monoiodosilver(I), Monotriphenyl phosphine monobromosilver(I), Monotriphenyl phosphine monochloro silver(I) and Monotriphenyl phosphine mononitro silver(I).

11. The process as claimed in claim 1, wherein said dialkyl ester of carboxylic acid is at least one selected from the group consisting of diethyl maleate, diisopropyl fumarate, diisopropyl maleate, and di-n-butyl maleate.

12. The process as claimed in claim 1, wherein said slow addition of said dialkyl ester of carboxylic acid in step (c) is over a time period in the range of 30 minutes to 150 minutes.
